(19) United States
(12) Patent Application Publication
(10) Pub. No.: US 2017/0055941 A1
(43) Pub. Date: Mar. 2, 2017

(54) INTEGRATED IMAGING COMPONENT AND INTRAVASCULAR DEVICE DELIVERY SYSTEM

(71) Applicant: KONINKLIJKE PHILIPS N.V., Eindhoven (NL)

(72) Inventors: Jeremy STIGALL, Carlsbad, CA (US); Princeton SAROHA, Ladera Ranch, CA (US); Maritess MINAS, San Diego, CA (US)

(21) Appl. No.: 15/255,914

(22) Filed: Sep. 2, 2016

Related U.S. Application Data
(60) Provisional application No. 62/213,561, filed on Sep. 2, 2015.

(51) Int. Cl. A61B 8/12; A61F 2/01; A61B 8/08; A61B 5/00; A61B 5/06

(52) U.S. Cl. CPC A61B 8/12; A61B 5/0066; A61B 5/065; A61B 8/5261; A61B 8/0841; A61F 2/01; A61F 2250/0096

(57) ABSTRACT

An apparatus and method for the intravascular placement of a filter includes an outer shaft with marker boards, an inner shaft, an intravascular ultrasound catheter with an ultrasonic imaging element, and the filter that is to be deployed. The ultrasound imaging element is enclosed within a proximal and distal transducer capsule configured to support the electronics of wiring of the element and provide for ease of insertion and withdrawal of the catheter. A guide wire is enclosed by and is moveable relative to the ultrasound catheter. In the stored position, the filter is secured adjacent to the distal inner shaft and proximal to the ultrasound catheter. When the apparatus is introduced into the vasculature, the ultrasound catheter provides real-time imaging of the vessel for identifying the appropriate location for placement of the filter. Once such a location has been identified, the filter is deployed and optionally, the ultrasonic imaging element used to confirm position, placement deployment of the filter.

### CROSS REFERENCE TO RELATED APPLICATIONS

This application claims the benefit of U.S. Provisional Application No. 62/213,561, filed Sep. 2, 2015.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The application relates to the use of intravascular imaging systems for positioning and retrieval of intraluminal devices, including for example, the use of ultrasound imaging for positioning and retrieval of endovascular filters and the like.

### BACKGROUND

The present invention relates to an apparatus and method enabling the use of intraluminal imaging components in cooperation with intraluminal devices for improvements to the implantation, positioning, deployment and retrieval of such devices. In one particular area of improvement is in the field of intravascular ultrasound-guided placement of vena cava filters, said filters often being necessary in the treatment of deep vein thrombosis.

A deep vein thrombosis is a medical condition wherein a blood clot, or thrombus, has formed inside a vein. Such a clot often develops in the calves, legs, or lower abdomen, but occasionally affects other veins in the body. This clot may partially or completely block blood flow, and, unlike clots in superficial veins, the clot may break off and travel through the bloodstream. Commonly, the clot is caused by a pooling of blood in the vein, often when an individual is bed-ridden for an abnormally long duration of time, for example, when resting following surgery or suffering from a debilitating illness, such as a heart attack or traumatic injury.

Deep vein thrombosis of the lower extremities is a serious problem because of the danger that the clot may break off and travel through the bloodstream to the lungs, causing a pulmonary embolism. This is essentially a blockage of the blood supply to the lungs that causes severe hypoxia and cardiac failure. It frequently results in death.

Placement of filters to assist in the prevention of clots traveling in the blood stream is usually accomplished using either the “femoral vein approach” or “jugular vein approach”, although alternative approaches, including “axiliary vein approaches”, may also be used. Many methods and approaches continue to use fluoroscopy for placement of a guide wire and catheter, as well as for placement and deployment of the filter. Such methods for placement and deployment of a filter also recommend the use of an intravenous dye with contrast angiography.

While some ultrasound based placement systems and methods have been described a need remains for improvements to intravascular placement of a filter that can be performed beside and thus does not necessitate the movement of the patient as well as for intravascular placement of a filter that will substantially reduce the overall time and cost of the placement procedure.

### SUMMARY OF THE DISCLOSURE

In general, in one embodiment, an intravascular imaging capsule, including an imaging component mounted on a support frame with a proximal end and a distal end having a base adapted and configured to at least partially encircle a support tube, a proximal facing shoulder mount and a distal facing shoulder mount; a distal tip having a distal end and a proximal end and a lumen extending from the proximal end to the distal end wherein a proximal portion of the lumen is sized to fit over a portion of the distal facing shoulder mount and a distal portion of the lumen is sized to have an inner diameter of about the same size as an inner diameter of the support tube; and proximal cap having a distal end and a proximal end and a lumen extending from the proximal end to the distal end sized to be greater than the largest cross section dimension of the support tube wherein a distal portion of the lumen is sized to fit over a portion of the proximal facing shoulder mount.

This and other embodiments can include one or more of the following features. In one aspect, the imaging component can be one of a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT). In another aspect, an exterior portion of the proximal cap can be scalloped to accommodate a portion of the imaging component. In a further aspect, the scalloped portion of the proximal cap can further include a pass through formed in a scalloped wall of the proximal cap extending from an opening in a proximal cap exterior wall to an opening in communication with the proximal cap lumen, the opening in the proximal cap exterior wall can be located distal to the opening in communication with the proximal cap lumen. In an alternative aspect, the scalloped portion of the proximal cap can further include a pass through formed in a scalloped wall of the proximal cap extending from an opening in a proximal cap exterior wall to an opening in communication with the proximal cap lumen, the pass through can form an angle of about 45 degrees relative to the proximal cap lumen. In yet another aspect, the portion of the imaging component can be an electronic connector for communication with an image processing system configured for use with the imaging component. In still another aspect, the electronic connect can include a flexible leg extending from the support frame. In another aspect, the proximal cap can further include a pass through formed in a wall of the proximal cap extending from an opening in a proximal cap exterior wall to an opening in communication with the proximal cap lumen, the opening in the proximal cap exterior wall can be located distal to the opening in communication with the proximal cap lumen. In a further aspect, the proximal cap can further include a pass through formed in a wall of the proximal cap extending from an opening in a proximal cap exterior wall to an opening in communication with the proximal cap lumen, the pass through can form an angle of about 45 degrees relative to the proximal cap lumen. In an alternative aspect, the pass through or opening can be sized to pass a microcable for connection of the imaging component to an image processing system configured for use with the imaging component. In yet another aspect, the capsule can further include a proximal portion of the proximal cap lumen sized to have an inner diameter to accommodate the support tube, a sheath over the support tube dimensioned to provide a gap between the sheath and the support tube to allow passage of a cable; and the sheath over the support tube can be attached to an interior wall of a portion of the proximal lumen. In still another aspect, the base can completely encircle the support tube. In another aspect, the base can have a cross section shape similar to a cross section shape of the support tube. In a further aspect, the cross section shape of the support tube can be circular. In an alternative aspect, the capsule can further include glue holes positioned to allow centering in the distal tip so that they can fill a gap between the support tube and an interior wall of the distal tip. In yet another aspect, the glue holes can be across a central longitudinal axis of the distal tip lumen. In still another aspect, the distal tip can have a tapered shape from the proximal end to the distal end. In one aspect, the distal most end of the distal tip can be a rounded atraumatic shape.

In general, in one embodiment, an image guided intravascular device delivery catheter, including a support tube with proximal end and a distal end and a lumen extending therethrough; a handle or y-valve having an aperture adapted to receive the proximal end of the support tube so that the lumen of the tube is coextensive with a lumen of the handle in communication with the handle aperture adapted to receive the support tube proximal end whereby movement of the handle produces movement of the support tube; a distal inner shaft having a proximal end and a distal end with a single lumen extending from the proximal end to the distal end sized to receive the support tube; a proximal inner shaft having a proximal end and a distal end with a first lumen and a second lumen each extending from the proximal end to the distal end wherein the first lumen is larger than the second lumen and the first lumen is sized to receive the support tube; an inner shaft marker attached to the proximal most end of the distal inner shaft or distal most part of the proximal inner shaft wherein the inner shaft maker is positioned to indicate the transition from the distal inner shaft to the proximal inner shaft; an outer shaft having a proximal end and a distal end and a lumen there through sized to receive the proximal inner shaft; an outer shaft marker positioned at the distal portion of the outer shaft; a locking device on the proximal portion of the outer shaft distal to the handle, the locking device having a locked configuration that impedes relative movement between the outer shaft and the handle and an unlocked configuration that permits relative movement between the outer shaft and the handle; an intravascular image tracking capsule having an imaging component supported between a distal tip and a proximal cap wherein the distal tip, the imaging component and the proximal cap are arranged about a common lumen that is adapted to receive the support tube distal end; and an intravascular device positioned between an exterior portion of the distal inner shaft and an interior portion of the outer shaft wherein while the outer shaft marker is positioned distal to the inner shaft marker movement between the intravascular device and the distal inner shaft is restricted.

This and other embodiments can include one or more of the following features. In one aspect, the device can further include a plurality of spaced apart distance markers along the outer shaft. In another aspect, the plurality of spaced apart markers can be printed on a surface of the outer shaft. In a further aspect, the plurality of spaced apart markers can indicate a distance relative to the intravascular device. In an alternative aspect, when the outer shaft marker is positioned proximal to the distal inner shaft marker relative movement between the intravascular device and the distal inner shaft can be permitted. In yet another aspect, when the outer6 shaft marker is positioned proximal to the distal inner shaft marker the intravascular device can be in a deployed configuration. In still another aspect, when the outer shaft marker is positioned proximal to the distal inner shaft marker the handle can be adjacent to the locking device. In one aspect, the axial distance moved by the outer shaft marker when moved from a position distal to the inner shaft marker to a position proximal to the inner shaft marker can be greater than an axial length of the intravascular device when positioned between an exterior portion of the distal inner shaft and an interior portion of the outer shaft. In another aspect, a proximal portion of the intravascular imaging capsule proximal cap can be adapted and configured to mitigate engagement with the intravascular device. In a further aspect, the intravascular imaging capsule proximal cap can have a tapered portion adjacent to where the support tube exits the intravascular imaging capsule. In an alternative aspect, the intravascular imaging capsule can be sized to pass through a portion of the intravascular device. In yet another aspect, the intravascular device can be a filter and the portion of the intravascular device can be a filtering component or a material capture structure. In still another aspect, the intravascular device can include one or more elements configured for detection by the imaging component. In one aspect, the one or more elements can be positioned on, about, along, around, or within one or more portions of the intravascular device such that when the one or more elements are detected by the imaging component an output of the imaging component can include an indication of a position, an orientation, a state of deployment, a state of retrieval, a state of operation, or a condition of the intravascular device. In another aspect, the imaging component can be one of a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT). In a still another aspect, the device can further include a communication cable connected at one end to the imaging element and at the other end to a connector adapted and configured for use with an image processing system configured for use with the imaging element, the communication cable passing through a lumen of the handle, the second lumen of the proximal inner shaft, along an outer surface of the distal inner shaft, and through an opening formed in the sidewall of the proximal cap. In an alternative aspect, the opening formed in the sidewall of the proximal cap can be angled towards the proximal portion of the distal inner shaft. In yet another aspect, the opening formed in the sidewall of the proximal cap can form an angle of about 45 degrees relative to an exterior wall of the proximal cap. In still another aspect, the opening formed in the sidewall of the proximal cap can be positioned in a portion of the proximal cap shaped to correspond to a portion of a flexible component coupled to the imaging element. In one aspect, the portion of the proximal cap shaped to correspond can have a smaller outer diameter compared to a directly adjacent portion of the proximal cap. In another aspect, the portion of the proximal cap shaped to correspond can have a flat surface that is sized to approximate a portion of the flexible component.

In general, in one embodiment, A method for positioning and deploying an intravascular device using an imaging device catheter, including advancing an imaging device and stowed intravascular device through a portion of the vasculature; assessing proximity of the intravascular device to a target site by evaluating a real time image data from the imaging device received during the advancing step; determining the intravascular device position within the vasculature of or near or in proximity to the target site or vessel using image data provided by the imaging device; and deploying the intravascular device within the vasculature based on the result of the determining step.

This and other embodiments can include one or more of the following features. In one aspect, the real time image data can be from an imaging device having an imaging component that can be one of a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT). In another aspect, the intravascular device can be one or more of a filter, a stent, a stent graft, a percutaneous valve, a prosthetic vascular component, and an implantable microfluidic device. In a further aspect, the method can further include before advancing step insert guidewire into the vasculature and advance to a target site. In an alternative aspect, the determining step may be used with a marking alone or with at least with one or more markings associated with the intravascular device. In yet another aspect, the determining step can be performed using an external visual marking on a sheath. In still another aspect, the determining step can be performed using a marking on the delivery system that can be perceptible by an imaging modality. In one aspect, the imaging modality can be an internal or external modality. In another aspect, the marking can be a radio opaque marker. In a further aspect, the marking can be on one or more of a distal inner tube and a sheath. In an alternative aspect, the marking can be an echogenic marker. In yet another aspect, the marking can include one or more markers and may be positioned on the intravascular device itself. In still another aspect, the method can further include after or during the deploying step repeating the determining step until the intravascular device is located in appropriate position relative to the target site. In a further aspect, wherein the determining step can be performed using data from the imaging device and a marker associated with the intravascular device. In one aspect, the marker associated with the intravascular device can be a distance marker on a sheath that is visible outside of the vasculature. In another aspect, the marker associated with the intravascular device can be on a delivery component or on the intravascular device and can be visible using an intravascular imaging modality, an external imaging modality or an imaging modality outside of the vasculature. In a further aspect, the marker associated with the intravascular device can be on the intravascular device and can be perceptible using an intravascular imaging modality, an external imaging modality or an imaging modality outside of the vasculature. In an alternative aspect, the marker associated with the intravascular device can be one or more elements positioned on, about, along, around, or within one or more portions of the intravascular device such that when the one or more elements are detected by an imaging modality an output of the imaging modality can include an indication of a position, an orientation, a state of deployment, a state of retrieval, a state of operation, or a condition of the intravascular device. In yet another aspect, the method can further include assessing or confirming a degree of intravascular device deployment, position of intravascular device relative to the target site or location of the intravascular device within the vasculature. In still another aspect, the step or assessing or confirming can be performed using an intravascular imaging modality, an external imaging modality or an imaging modality outside of the vasculature. In a further aspect, the step or assessing or confirming can be performed by analysis of image data of one or more markings associated with the intravascular device. In one aspect, the imaging device can be adapted and configured to pass through a portion of the intravascular device when the intravascular device is in the deployed configuration.

In general, in one embodiment, A method of positioning and deploying an intravascular device within the vasculature of a patient, including advancing an imaging device and stowed intravascular device through a portion of the vasculature; assessing proximity of the intravascular device to a target site by evaluating a real time image data from the imaging device received during the advancing step; determining the intravascular device position within the vasculature of or near or in proximity to the target site or vessel using image data provided by the imaging device; and deploying the intravascular device within the vasculature based on the result of the determining step.

This and other embodiments can include one or more of the following features. In one aspect, the assessing step, the determining step or the deploying step can be performed without the use of an external imaging modality. In another aspect, the assessing step, the determining step or the deploying step can be performed in a patient hospital room. In a further aspect, the assessing step, the determining step or the deploying step can be performed without exposing the patient to a radiation source. In an alternative aspect, the assessing step, the determining step or the deploying step can be performed by identification of an anatomical landmark within the body. In yet another aspect, the anatomical landmark can be within the vasculature. In still another aspect, the vasculature can be an artery. In one aspect, the vasculature can be a vein. In another aspect, the anatomical landmark can be a change in size of a vessel. In a further aspect, the anatomical landmark can be a junction of two vessels. In an alternative aspect, the anatomical landmark can be the junction of a renal vein and a vena cava.

In yet another aspect, the anatomical landmark can be an iliac bifurcation. In still another aspect, the anatomical landmark can be a pre-selected diameter of a vena cava. In a further aspect, the anatomical landmark can be a pre-selected diameter of the vessel based on a deployed configuration of the intravascular device. In one aspect, the assessing step, the determining step or the deploying step can be performed by identification of a marker associated with the intravascular device that is perceptible using the intravascular imaging modality of the imaging device. In another aspect, the marker associated with the intravascular device can be one or more elements positioned on, about, along, around, or within one or more portions of the intravascular device such that when the one or more elements are detected by an imaging modality an output of the imaging modality can include an indication of a position, an orientation, a state of deployment, a state of retrieval, a state of operation, or a condition of the intravascular device. In a further aspect, the method can further include assessing or confirming a degree of intravascular device deployment, position of intravascular device relative to the target site or location of the intravascular device within the vasculature using imaging data provided by the imaging device. In an alternative aspect, the step or assessing or confirming can be performed using an intravascular imaging data provided by an imaging component can be one of a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT). In yet another aspect, the imaging device can be adapted and configured to pass through a portion of the intravascular device when the intravascular device is in the deployed configuration.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates the general procedures for the use of an embodiment of the inventive imaging catheter and intravascular device delivery technique for placement of the intravascular device within the vasculature.

FIG. 2A is an overview of an embodiment of an inventive ultrasound catheter and vena cava filter deployment system in a closed or filter loaded configuration.

FIG. 2B is an overview of the ultrasound catheter and filter deployment system of FIG. 2A in an open/filter deployed configuration where the filter has been separated from the delivery catheter.

FIG. 3A is a perspective view of an embodiment of the imaging component capsule at the distal most portion of an embodiment of an imaging catheter for delivery of an intravascular device.

FIG. 3B is a cross section view of the imaging component capsule in FIG. 3A.

FIGS. 4A and 4B are perspective and cross-section views respectively of the distal tip portion of the intravascular imaging component capsule of FIGS. 3A and 3B.

FIGS. 5A and 5B are perspective and cross section views respectively of the proximal cap portion of the intravascular imaging component capsule of FIGS. 3A and 3B.

FIGS. 6A and 6B illustrate an alternative embodiment of the distal tip position (FIG. 6A) and the proximal cap portion (FIG. 6B) of an intravascular imaging component capsule adapted and configured for use with non-round imaging component support frames.

FIG. 7 is a cross-section view of a dual lumen shaft.

FIG. 8 is an exterior view of a portion of a dual lumen shaft having linear markings spaced along the exterior with a portion of a distal inner shaft extending from the distal end.

FIG. 9 is a cross-section view of a coaxial shaft assembly formed by a support tube and a sheath forming a gap dimensioned to form a passage for a component such as an imaging component cable.

FIG. 10 is a perspective view of a Y-arm hub design.

FIG. 11 is an exemplary inventive method of positioning and displaying a filter using embodiments of the ultrasound catheter described herein.

FIG. 12 is a cross section view of an exemplary loading configuration of an intravascular device in a stowed configuration relative to the distal inner shaft of an imaging component catheter embodiment.

FIG. 13 is a cross section view of an exemplary loading configuration of an intravascular device in a stowed configuration relative to the distal inner shaft of an imaging component catheter embodiment whereby the distal inner shaft passes through a portion of the intravascular device here illustrated as a portion of the inner shaft within one or more spiral frames of a filter.

FIG. 14 is a cross section view of an exemplary loading configuration of an intravascular device in a stowed configuration relative to the distal inner shaft of an imaging component catheter embodiment whereby the intravascular device is in a stowed configuration within a separate storage cartridge alongside the distal inner shaft.

FIG. 15A is a perspective view of the distal end of an integrated ultrasound transducer filter delivery catheter where the handle is in an open configuration and the filter is in a stowed configuration.

FIG. 15B is a perspective view of a more proximal portion of the distal end shown in FIG. 15A of the integrated ultrasound transducer filter delivery catheter where the filter and marker relationship is illustrated with the handle remaining in an open configuration and the filter is in a stowed configuration.

FIG. 15C is a perspective view of the distal end shown in FIGS. 15A and 15B of the integrated ultrasound transducer filter delivery catheter where the handle has been closed (FIG. 15D) to permit movement between the distal inner tube and the outer tube and transition the filter from stowed to deployed configuration. Also illustrated in the portion of the distal inner tube that remains through a portion of the filter. The distal inner tube is shown extending through a material capture structure and support frame.

FIG. 15D is a perspective view of the handle at the proximal end of the catheter in the condition of FIG. 15C (filter transitioned from stowed to deployed configuration).

FIG. 15E is a perspective view of the integrated imaging delivery catheter of FIG. 15C after withdrawal of the imaging capsule through the filter.

### DETAILED DESCRIPTION

In various alternative embodiments of aspects of this invention there is provided an integrated intravascular imaging guidance and delivery catheter designed to allow a user to accurately and safely deploy an intravascular device, in one aspect, using only the image guidance provided by the system. In the various alternative embodiments, the catheter system is adapted and configured for use with any of a variety of imaging components, such as, for example, a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT). Various details of imaging components may be provided by reference to U.S. Pat. No. 6,899,682 and U.S. Pat. No. 5,938,615, each of which are incorporated herein by reference in its entirety for all purposes. Additional aspects of an embodiment of the present invention adapted for rotational imaging components may be appreciated through reference to U.S. Pat. No. 8,403,856, incorporated herein by reference in its entirety for all purposes.

In one aspect, the intravascular image guidance catheter system also retains a functional compatibility with angiography. In one embodiment, the intravascular image guided delivery system is adapted and configured for delivery of any of a wide variety of intravascular devices such as stents, grafts, filters, monitoring devices, implantable devices, prosthetics of portions of the vasculature such as valves in any of the arterial or venous trees, for example. In one embodiment, the cooperation of the various elements in the imaging component enabled delivery catheter advantageously permit a physician to determine readily how far along a delivery pathway the intravascular device and/or a designated distal portion of the delivery system resides within or relative to the patient anatomy in relation to a targeted delivery or interventional site. Moreover, the distal tip section of the imaging component enabled catheter device was designed to reduce the risk of intravascular device drag post deployment and ingress related imaging component image failures.

In various alternative embodiments of aspects of this invention there is provided an integrated intravascular ultrasound guidance and delivery catheter designed to allow the user to accurately and safely deploy a Vena Cava Filter guided only by a build in (internal) IVUS system. In one aspect, the intravascular ultrasound guidance catheter system also retains a functional compatibility with angiography. In one embodiment, the intravascular ultrasound guided delivery system is adapted and configured for delivery of a filter developed by Crux Biomedical, Inc. as described in international patent application WO2014/152217 having International Application Number PCT/US 2014/027083. In one embodiment, the cooperation of the various elements in the ultrasound enabled delivery catheter advantageously permit a physician to determine readily how far along a delivery pathway the filter and/or a designated distal portion of the delivery system resides within or relative to the patient anatomy. Moreover, the distal tip section of the ultrasound enabled catheter device was designed to reduce the risk of filter drag post deployment and ingress related IVUS image failures.

The various embodiments of the imaging component enabled catheter delivery system provide numerous advantages over conventional filter delivery systems such as, by way of nonlimiting examples:
Deployment of a Vena Cava Filter using only IVUS guidance.
Post deployment IVUS inspection of deployed filter wireforms and webbing.
Improved ease and convenience of medical procedure. Procedure can be done bedside instead of an angiography lab.
Improved patient outcomes. Shortened procedure times, and patients are exposed to less radiation compared to the common angiography practice.
Linear markings on shafts inform the user how far they are relative to the area being imaged by IVUS.
Filter deployment workflow has been characterized and optimized for accuracy relative to the imaging plane of the transducer. Reduced risk over the current practice of deploying a Vena Cava Filter under IVUS guidance only.
Capsulation of IVUS transducer through the unique design of the tracking tip and transducer support. Mitigates the risk of both physically induced and ingress related image failures.
   While the examples above single out an IVUS transducer, the advantages above may also be achieved by various other embodiments of integrated imaging catheters with other imaging components as described herein.

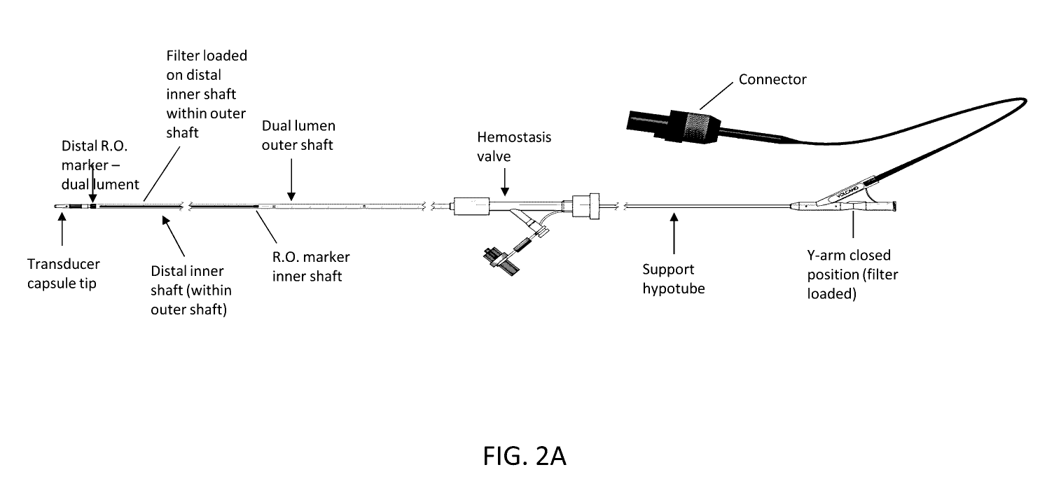

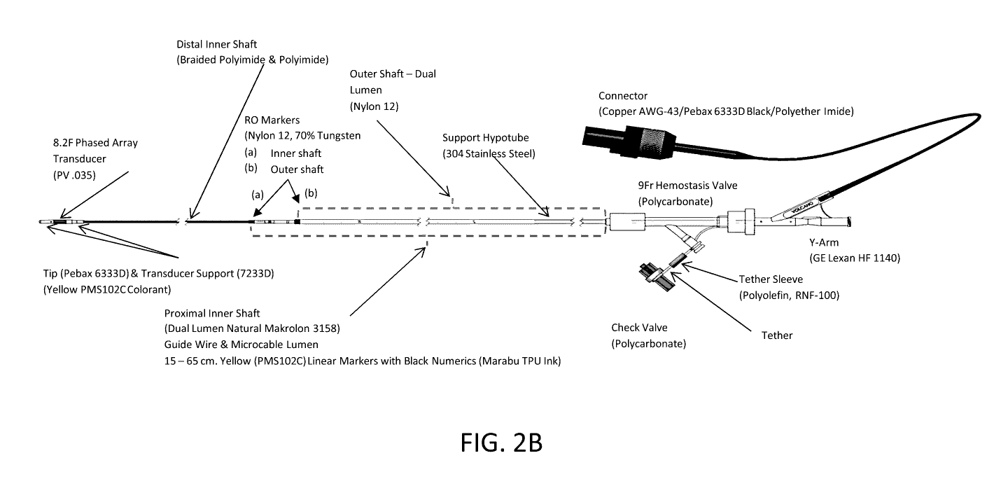

By way of introduction, a better understanding of the overall apparatus 40 of an embodiment of the present invention can be achieved through a review of the function and operation of the apparatus 40, the details of which are illustrated in FIGS. 2A and 2B. Additional general aspects of image guided delivery into the vena cava may be appreciated through reference to U.S. Pat. No. 6,440,077 and U.S. Pat. No. 6,645,152, each of which is incorporated herein by reference in its entirety for all purposes.

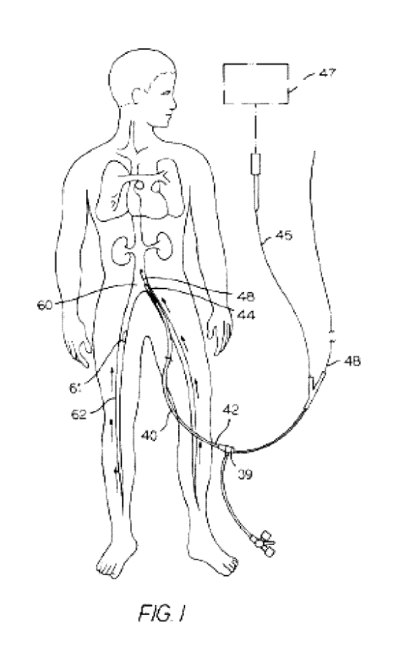

Referring first to FIG. 1, a thrombus 61 is present in the right femoral vein 62. As indicated by the arrows, blood flows from the femoral veins 62 through the vena cava 60 toward the heart and lungs, and thus placement of a vena cava filter 50 (not shown) is necessary to prevent the thrombus 61 from traveling to the heart and lungs should it break free.

To deploy the vena cava filter 50 using the apparatus 40, the guide wire 48 is inserted into the vena cava 60, preferably using the Seldinger technique from the femoral position. Over the guide wire 48, the outer sheath 42 and IVUC 44 are passed percutaneously through the femoral vein 62 and into the vena cava 60. In this regard, there is preferably a coupling, as indicated by reference numeral 39 in FIG. 1 that is secured to a distal end of the outer sheath 42. This coupling has an internal seal (not shown) that maintains the positions of the outer sheath 42 and the IVUC 44 relative to one another, thus allowing the outer sheath 42 and the IVUC 44 to be moved together as a unit. When it is necessary to move the IVUC 44 relative to the outer sheath 42, the attending physician must physically maintain the position of the coupling 39 while manually advancing the IVUC 44 through the coupling 39; or, the attending physician may physically maintain the position of the IVUC 44 while drawing back the outer sheath 42 and coupling 39 relative to the IVUC 44. In various embodiments, the action of moving a stowed intravascular device on the integrated catheter delivery system into a deployed condition is achieved by relative movement between the outer dual lumen shaft and the distal inner sheath via movement of the handle as discussed below.

As the ultrasound transducer imaging capsule travels through the femoral vein 62 and into the vena cava 60, the ultrasonic imaging element 46 of the IVUC 44 provides a real-time ultrasonic picture of its passage, thereby assuring proper placement of the filter 50. In this regard, the ultrasonic signals received by the ultrasonic imaging element 46 are transmitted through internal wiring 45 of the IVUC 44 to an external signal processor (as indicated in phantom and by reference numeral 47 in FIG. 1). This real-time imaging of the veins also allows for measurement of the inner diameter of the vena cava 60 and provides a visual confirmation that there is no thrombus in the area selected for deployment of the filter 50. Finally, the ultrasonic imaging allows for extremely accurate identification of the position of the renal veins 64, 66 to further ensure appropriate placement of the filter 50. In this regard, it is preferred that the outer sheath 42 and IVUC 44 are moved through the vena cava 60 past the renal veins 64, 66 so that the attending physician can view the portions of the vena cava 60 adjacent the renal veins 64, 66. The outer sheath 42 and IVUC 44 are then drawn back to an appropriate position below the renal veins 62, 64 for deployment of the filter 50. After deployment of the filter 50, the preferred apparatus 40, sans the filter 50, is withdrawn from the vena cava 60.

FIG. 2A is an overview of an embodiment of an inventive ultrasound catheter and vena cava filter deployment system in a closed or filter loaded configuration.

FIG. 2B is an overview of the ultrasound catheter and filter deployment system of FIG. 2A in an open/filter deployed configuration where the filter has been separated from the delivery catheter.

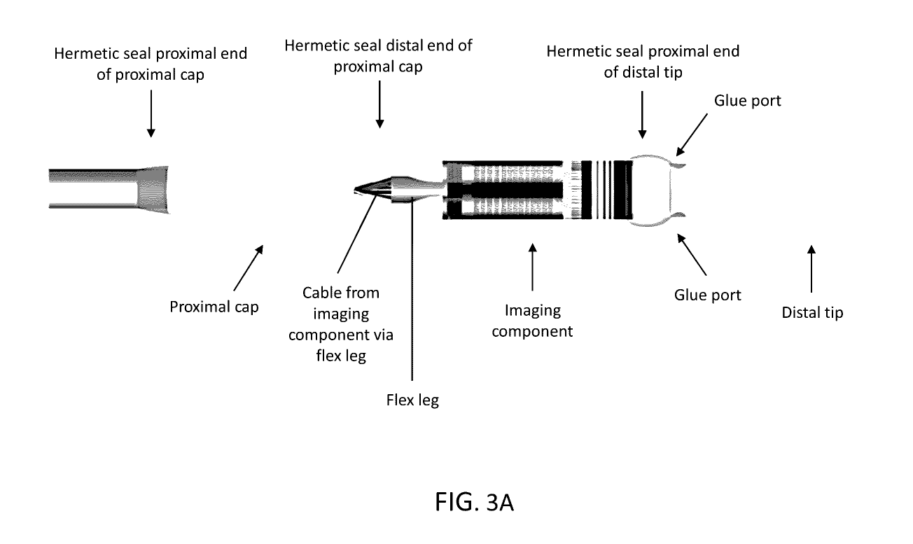

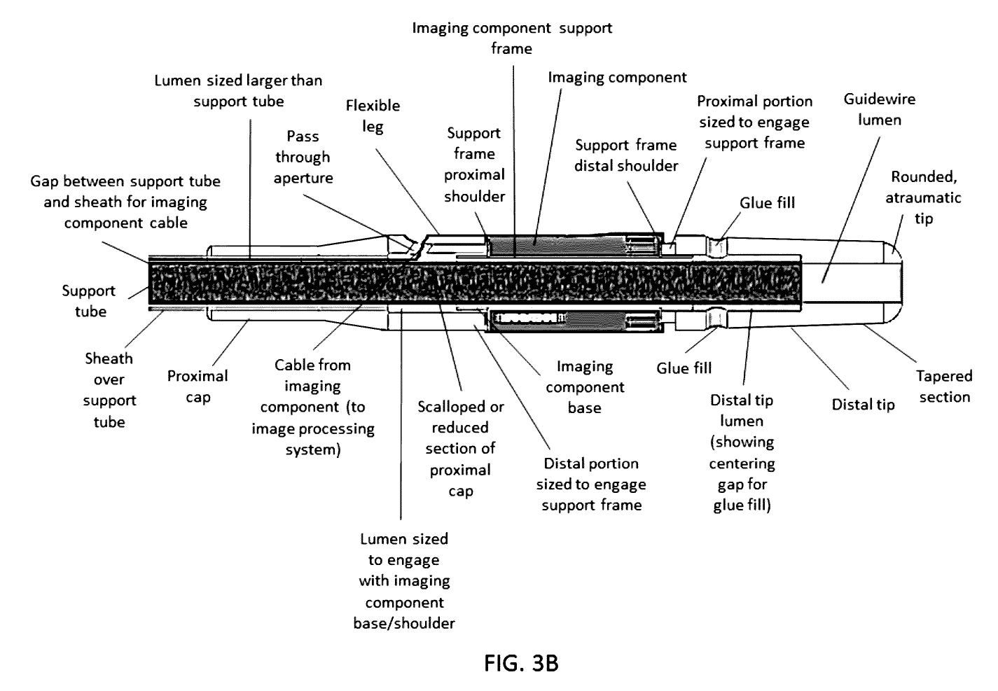

FIG. 3A is a perspective view of an embodiment of the imaging component capsule at the distal most portion of an embodiment of an imaging catheter for delivery of an intravascular device.

FIG. 3B is a cross section view of the imaging component capsule in FIG. 3A.

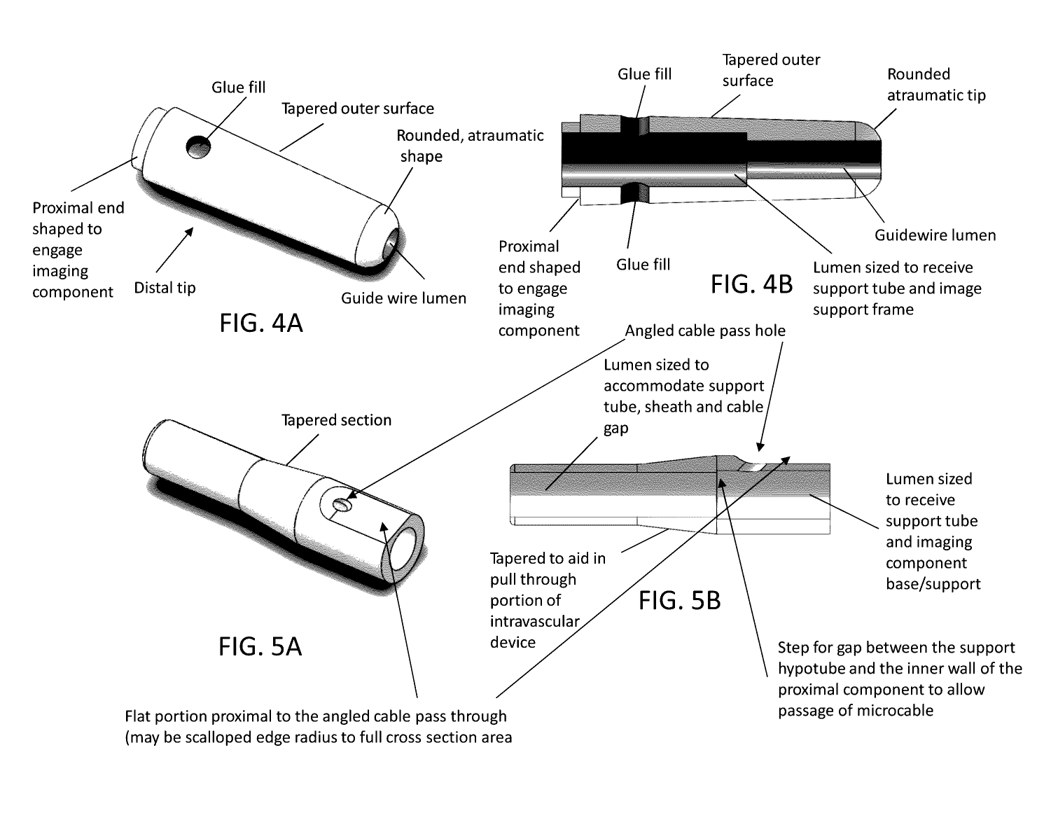

FIGS. 4A and 4B are perspective and cross-section views respectively of the distal tip portion of the intravascular imaging component capsule of FIGS. 3A and 3B. In this aspect of the novel tracking tip design, the tip portion has been manufactured through either physically altering the extruded raw material or injection molding. As best seen in FIG. 4A, the tip is designed with a beveled distal entry, allowing the catheter to glide easily and safely when navigating to the target site. This atraumatic tip design combined with the stiff material selected allows the device to be used bareback (inserted without a guide catheter), without causing unwanted trauma to the patient or damage to the device.

As best seen in FIG. 4B there are two glue port holes make it convenient to flow adhesive through and create a strong bond between the tracking tip and the inner member. The proximal end of the tip is designed with a narrow step that provides a circumferential slot for adhesive filling. This conveniently allows the adhesive to bond the tip with the adjacent stiff tubular body of the transducer, preventing dislodgment, without adding excessively to the overall outer diameter. The internal cavity of the tracking tip is designed with a stopper to precisely terminate the advancement of the tubular body of the transducer and creates an equal and concentric inner cavity between the two parts to ensure smooth guide wire movement. In some embodiments, a step or shoulder is provided within the tip so dimensioned as to provide a termination as the desired location within the capsule tip.

FIGS. 5A and 5B are perspective and cross section views respectively of a proximal end portion of an intravascular ultrasound transducer support capsule of FIGS. 3A and 3B. Aspects of ultrasound transducers may be appreciated by reference to U.S. Pat. No. 4,917,097 and U.S. Pat. No. 5,938,615, each of which are incorporated herein by reference in its entirety. In one aspect, the novel transducer support design has been manufactured through either physically altering the extruded raw material or injection molding. The support is designed with a step in its inner cavity as best seen in FIG. 5B. The step provides structural support for the tubular body of the transducer on the distal end and the inner member on the proximal end. In one embodiment, the step is dimensioned so that the tubular body of the transducer will terminate just prior to the entry hole on the distal end when assembled. In one aspect, the pass through aperture is a 45 degree angle hole relative to the longitudinal axis of the transducer capsule. This pass through aperture is adapted and configured for routing one or more cables connected to the imaging component, along the flexible leg, through the provided gap to a more proximal structure. In one aspect, the transducer support capsule is adapted and configured for use with imaging component designs that employ one or more microcables as needed by a particular imaging component. In one aspect, a suitable transducer array or imaging component would include, for example, a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT). As such, in one aspect, the transducer support capsule is adapted and configured for use with an ultrasound transducer using microcables.

As used herein, a microcable is sized from 0.005-0.010 inches, and may include one or more individually insulated wires. In one embodiment, the number and type of wires provided in a microcable is selected to correspond to the number of cables needed to provide electrical connectivity and communication between each of the components in an embodiment of the intravascular imaging capsule. In various specific embodiments, the number of individual wires forming a microcable can be 2, 3, 4, 5, 6, 7, 8, 9, 10 or more depending upon specific configurations or functionalities provided. In one embodiment the wires provided in the microcable are arranged in a twist configuration. In one specific aspect, there are seven individually insulated wires in a twisted configuration provided as a microcable for use with the intravascular imaging capsule.

It is to be appreciated that based on the specific imaging component configuration the overall size and orientation of the pass through aperture in the intravascular proximal cap will vary depending on the size, number and configuration of the wires used and the resulting microcables arrangement. Moreover, the gap or spacing between the sheath over the support tube and the support tube and the length and orientation of the flexible leg are also adapted and configured and subject to modification in accord with the specific imaging component and capsule configuration employed.

The use of the inner aspects of both the transducer capsule as well as the gap in the shaft components allow for great ease of manufacturability and durability of microcable integration and assembly. Manual notching and later sealing steps are eliminated along with the related potential for premature device failure or hermetic seal leaks. Still further, the cooperation permitted by the incorporation of the flat portion of the proximal cap and scalloped cut out permit the position of the angled aperture and flexible leg to and in reducing stresses while transitioning the microcable from inside the lumen into the intravascular capsule and imaging component. The beneficial arrangement of the components above may be appreciated with reference to FIGS. 3B, 5A and 5B. In one embodiment, the transducer support capsule permits the run of the microcable out of the inner lumen of the transducer support in a manner that readily permits welding. In still another aspect, the step was also designed to insure that the inner lumens of the two parts create an equal and concentric pathway for smooth guide wire movement.

As best seen in FIGS. 5A and 5B, the outer proximal surface of the transducer capsule is designed to have an atraumatic proximal end (e.g. soft gradual taper) for ease of retraction during procedural use. The transducer capsule proximal portion also includes a flat cut out on the same surface as the exit for the angled cable hole on the distal end of the component (see FIGS. 5A/5B). The flat cut out section provides structural support against kinking and ample room to pad the transducer flex leg with adhesive, further mitigating the risks of physically induced or ingress related image failures.

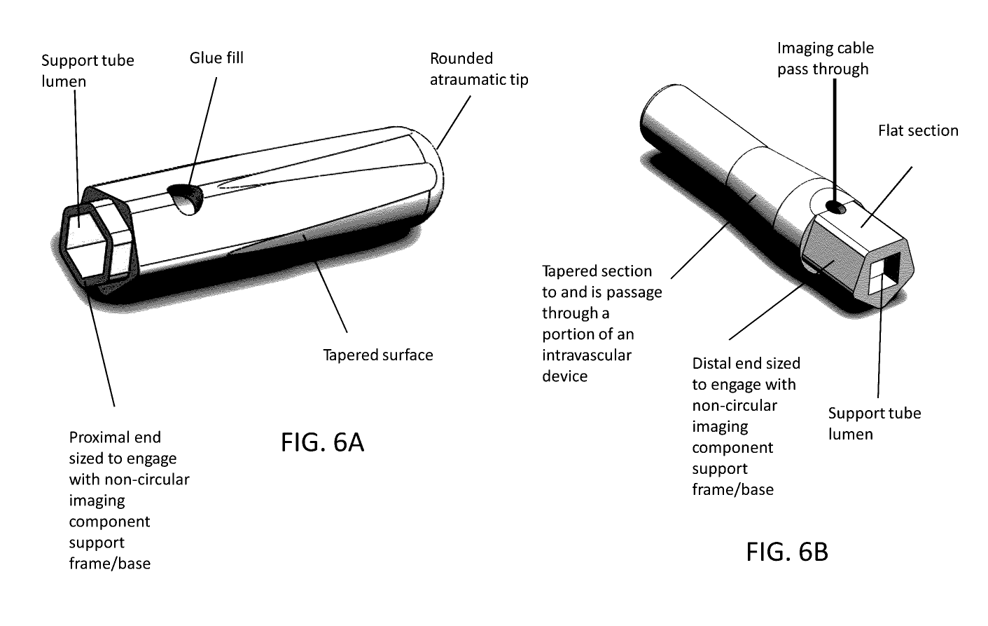

FIGS. 6A and 6B illustrate an alternative embodiment of the distal position (FIG. 6A) and the proximal portion (FIG. 6B) of an ultrasound transducer capsule as shown in FIGS. 4A and 5A. The illustrative embodiments of FIGS. 6A and 6B illustrates distal tips and proximal transducer support structures having various shaped inner diameters/outer diameters at junction ends. By way of example, FIG. 6A illustrates a distal tip having a hexagon shaped junction end. FIG. 6B illustrates a pentagon shaped junction on the proximal capsule transducer support structure. In these alternative embodiments, there are provided keyed inner diameters to match the offset shape of the adjacent component (e.g., the transducer unibody best seen in FIGS. 3A and 3B). In still other aspects, there is provided matching outer diameters (i.e., matching as to shape and dimension) so as to create a flushed profile at the junction between the transducer unibody portion and the proximal or distal component thereby promoting a strong hermetic seal if needed.

The transducer unibody, in some embodiments, refers to the assembly having an imaging component mounted on a support frame with a proximal end and a distal end having a base adapted and configured to at least partially encircle a support tube. There is also a proximal facing shoulder mount and a distal facing shoulder mount. The proximal and distal shoulder mounts may take on various different shapes, sizes and configurations based on the respective overall capsule design as well as the mating interfaces on the distal tip, the proximal cap and the overall design requirements of the imaging capsule. In some embodiments, one or both of the inner diameter and outer diameter shapes can be held throughout the entire length of the component or transition (step or gradual) to a different shape for functional use (e.g. shapes to match a second adjacent component or rounded off to create an atraumatic tip).

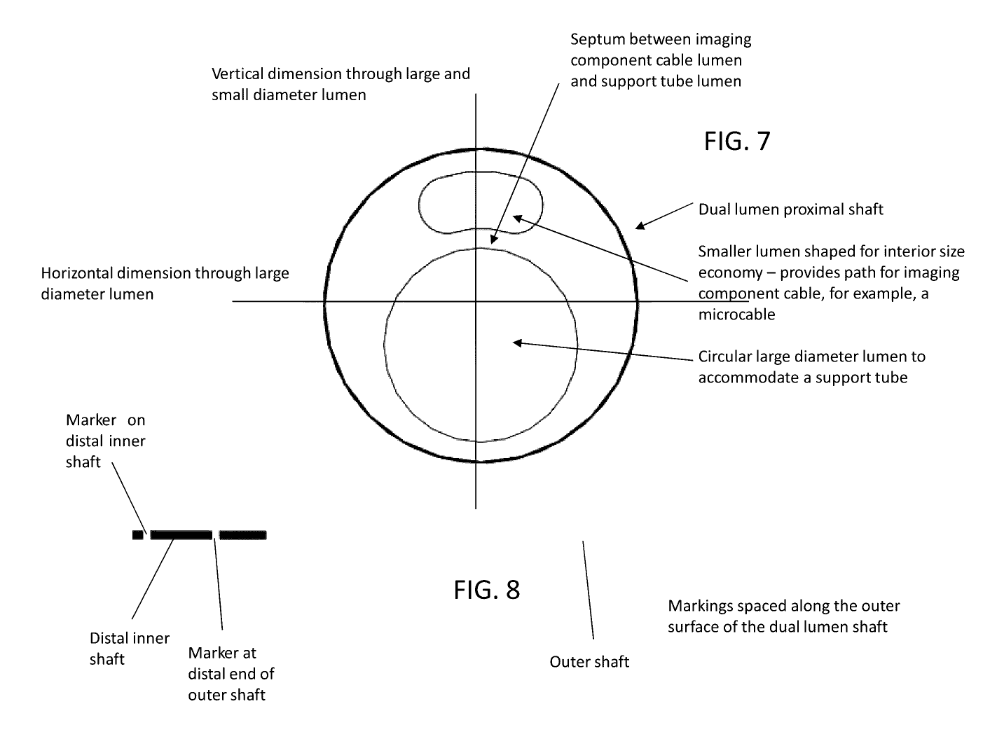

FIG. 7 is a cross-section view of a dual lumen shaft and an exterior view of the shaft with linear markings. The larger lumen is a circular lumen and is vertically centered with the smaller lumen. The smaller lumen has the profile shown in FIG. 7 above to conserve space. The unique profile allows the shaft to be extruded with larger wall thicknesses and lumens despite minimal increases in the overall outer diameter. The illustrated shape is a section of the outer perimeter beyond the inner lumen. The illustrated shape has rounded ends with curved upper and lower sides generally forming a kidney shape or curved cresent shaped oval. The primary function of the smaller lumen in the dual lumen portion of the outer shaft is the safe transport of the microcable from one end of the outer shaft to proximal end and on to the connector. The larger lumen does the same for a guide wire and the support hypotube.

The dual lumen shaft may be formed from a number of suitable materials including for example, materials evaluated for this design: Nylon, Pebax (various grades 55D-72D), Polycarbonate, Braided SST hypotube, and PEEK.

In one embodiment the larger lumen is formed by a braided polyimide sized to receive the support tube. In another aspect, the smaller lumen is provided by a polyimide or is a braided polyimide. The outer layer of the dual lumen structure may be a Pebax/nylon compound or FEP. In one specific embodiment, the large lumen is a braided polyimide, the small lumen is a polyimide and the outer layer is a PEBAX/nylon. In one specific embodiment, the large lumen is a braided polyimide and the outer layer is FEP. Approximate dimensions of the dual lumen shaft horizontal across the large lumen and vertical through the small lumen are 0.053″ and 0.058″ in a first embodiment; 0.0715″ and 0.078″ in a second embodiment and 0.0515″ and 0.056″ in a third embodiment. In still further variations, the wall or septum between the microcable lumen and the support tube lumen is between about 0.0035-0.010 inches. In one specific embodiment, the dual lumen shaft has a minimum wall thickness of at least 0.0035 inches, is made of polycarbonate and includes a crescent oval shaped microcable lumen oriented towards the support tube conduit as in FIG. 7.

The outer surface of the dual lumen shaft also features a linear marking design as illustrated in FIG. 8. The markings are provided using any suitable method such as labels, etching, laser marking or applied onto the shaft through a pad printing process. The origin of the markings is set based on the specific configuration of the catheter, the imaging component and the intravascular device. In one embodiment, the zero mark or a reference mark for the markings on the outer shaft is set on a portion of the intravascular device. In one embodiment, the zero mark or a reference mark for the markings on the outer shaft is set on a portion of the intravascular imaging device. In one embodiment, the zero mark or a reference mark for the markings on the outer shaft is set on a portion of the imaging device capsule. In one aspect, the markings are linearly spaced out according to real measurement units (e.g. 1 cm apart) from one another and printed circumferentially throughout the shaft's length. Text can also be added on top of these markings indicating either their measurement equivalent or some sort of notification/warning. During assembly, the first linear mark on the distal end is lined up with the imaging plane of the IVUS transducer or imaging device. Alignment with the IVUS imaging plane creates a reliable reference for deployment workflow and a convenient way for the user to know how far from the entry site is the IVUS imaged area as well as a tool to make accurate quantitative observations.

FIG. 8 is an exterior view of a dual lumen shaft having linear markings spaced along the exterior. The marking may be printed on an exterior sheath over the dual lumen shaft. The markings may have major and minor linear distance markings such as major units of 5 cm increments and minor markings of 1 cm increments. Other units of measure and different major and minor divisions may be provided. Also shown in this view is a portion of the distal inner shaft extended beyond the distal end of the dual lumen shaft. Two additional markers are also shown in this view. There is a marker on the distal inner shaft and a marker on the distal most end of the dual lumen shaft. In one embodiment the marker on the distal inner shaft is positioned in relation to the intravascular device when the device is stowed against the distal inner shaft. In one embodiment, the distal inner shaft marker is positioned in relation to the distal most end of the intravascular device. In one embodiment, the distal inner shaft marker is positioned in relation to the proximal most end of the intravascular device. In one embodiment, the distal inner shaft marker is positioned in relation to a portion of the intravascular device to be positioned in a specific location within the vasculature or in a target location. The position of each of the markers on the distal inner shaft or the outer dual shaft may be selected based on the procedure being performed using the image guided catheter described herein or the location for use of the intravascular device. The materials used for the markings may be selected to be detected by the intravascular imaging system or an exterior imaging system.

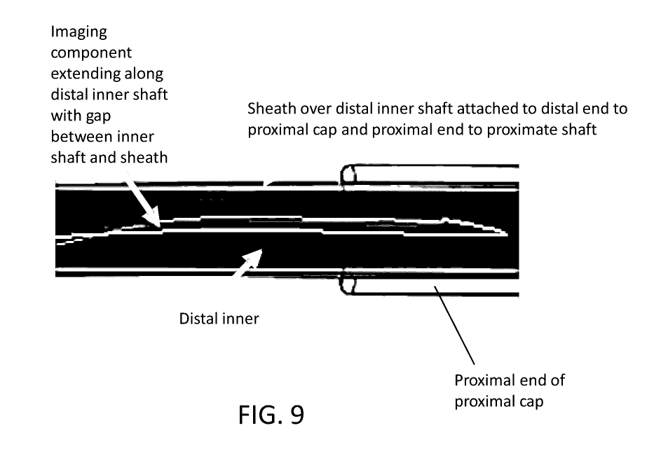

FIG. 9 is a cross-section view of the coaxial shaft assembly. In this enlarged section view this is shown the relationship of the sleeve, shaft and microcable. The sleeve and the shaft are arranged to function as two coaxial shafts. In one embodiment of this dual shaft design there is a more rigid shaft to be jacketed over by a thinner and more pliable shaft or sleeve. The dimensions of these shafts are intentionally specified to leave a small gap when assembled coaxially, allowing a component (e.g. microcable) to pass through between them free from damage. The gap ranges from about 0.01-0.003″ or about 0.0254-0.1016 mm. The two shafts of this assembly are only fixed on the proximal and distal most ends. In one embodiment the sheath is attached to the proximal cap of the imaging capsule and to the distal end of the inner shaft. As a result, the section in between the joined proximal and distal portions and the component it contains are allowed to free float along the unattached portion. The movement of this section reduces the stress exerted on the components when the assembly is bent. In still other aspects, an additional shaft can be used to encase the microcable component during insertion between the shaft and sleeve to create a gradual transition in lumen durometer and further mitigate any risk of kinking.

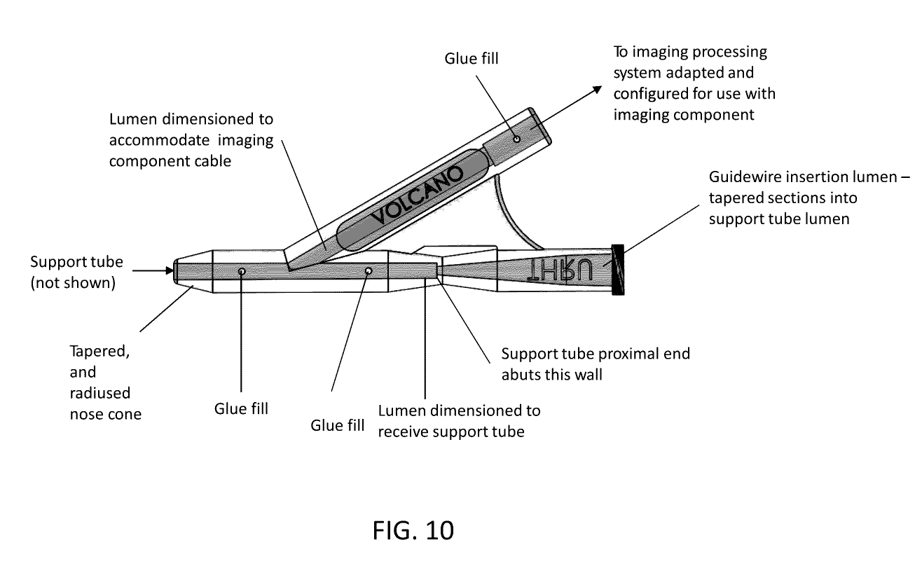

FIG. 10 is a perspective view of a Y-arm hub design. This design provides improvements over other conventional Y-arm designs. For example, the hub features corporate branding on the side branch surface, and the “THRU” text on the proximal end of the guide wire port. The inner cavity of the hub features the traditional straight side branch used to route the microcables, but now also features two oppositely oriented tapered sections for guide wire insertion. The two oppositely oriented tapered sections are separated by a step, which also functions to terminate the proximal inner shaft and concentrically align its lumen with the proximal guide wire cavity of the hub for smooth guide wire movement. This design features 3 glue port holes, one thru all side branch hole and two thru surface distal guide wire cavity holes. The thru surface holes in the distal cavity was designed for manufacturing process versatility, improved ease and consistency related to the use of low viscosity adhesive for bonding parts. Lastly, the distal end of the Y-Arm hub was shortened and shaped into a tapered and radiused nose cone, eliminating the need for a polyolefin cover for cosmetic and radiusing purposes.

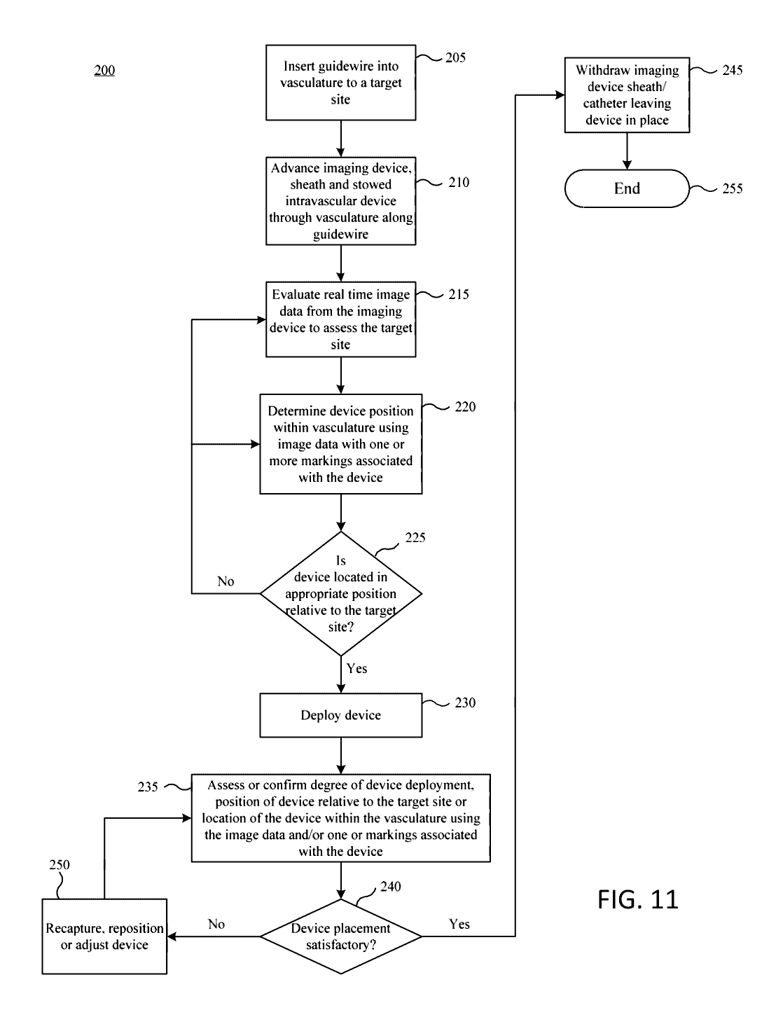

FIG. 11 illustrates a method 200 for positioning and deploying an intravascular device using an imaging device catheter. First, at step 205, insert guidewire into the vasculature and advance to a target site. The use of a guidewire may be optional in some embodiments, with some catheters or procedures. Next, at step 210, advance imaging device, sheath and stowed intravascular device through vasculature along guidewire. Next, at step 215, evaluate real time image data from the imaging device to assess the target site. Next, at step 220, determine intravascular device position within the vasculature of or near or in proximity to the target site or vessel using image data provided by the imaging device alone or with one or more markings associated with the intravascular device. A marking associated with the imaging device may include, for example, the use of an external visual marker on a sheath (see FIG. 8), a marker on the delivery system that is perceptible by an imaging modality including an internal or external modality (see radio opaque markers on the distal inner tube and the sheath in FIGS. 2 and 8 for example). Additionally or alternatively, the one or more markers may be positioned on the intravascular device itself such as described in International PCT Publication No. WO 2013/106746 A2, titled “ENDOLUMINAL FILTER WITH FIXATION;” International PCT Publication No. WO 2014/152217 A1, titled “ENDOLUMINAL FILTER HAVING ENHANCED ECHOGENIC PROPERTIES;” International PCT Publication No. WO 2014/152365 A2 titled “FILTERS WITH ECHOGENIC CHARACTERISTICS,” and “ENDOLUMINAL FILTER HAVING ENHANCED ECHOGENIC PROPERTIES” U.S. Provisional Patent Application Ser. No. 62/054,844 filed Sep. 24, 2014, each of which are herein incorporated by reference in its entirety. It is to be appreciated the filter being deployed using the systems and methods described herein may be secured or controlled using the methods, devices or techniques described in U.S. Provisional Patent Application No. 61/919,573, titled “SECUREMENT DEVICE FOR CONTROLLED ENDOLUMINAL FILTER DEPLOYMENT,” filed Dec. 20, 2013 and U.S. patent application Ser. No. 14/578,087, titled “DEVICES AND METHODS FOR CONTROLLED ENDOLUMINAL FILTER DEPLOYMENT,” filed Dec. 19, 2014 incorporated herein by reference.

Next, at step 225, is intravascular device located in appropriate position relative to the target site? If the answer to step 225 is “NO” then proceed to perform one or both of steps 215 and 220 until the answer to step 225 is “YES”. If or when the answer to step 225 is “YES” then proceed to step 230 and deploy the intravascular device. Next, at step 235, assess or confirm degree of intravascular device deployment, position of intravascular device relative to the target site or location of the intravascular device within the vasculature using the ultrasound image data and/or one or markings associated with the intravascular device. Thereafter, continue to step 240 and determine whether or not the intravascular device placement is satisfactory. If the answer to step 240 is ‘NO’ then proceed to step 250 to recapture, reposition or adjust the intravascular device position. Thereafter, return to step 235 and assess the intravascular device deployment. If the answer to step 240 is “YES”, then proceed to withdraw U/S transducer sheath/catheter leaving intravascular device in place. Once complete the method ends at step 255. Once complete the method ends at step 255.

By way of a specific example, FIG. 11 will be described for an image guided delivery where the intravascular device is a filter. More specifically, this is a description of a specific embodiment of the method 200 for positioning and deploying a filter using an ultrasonic imaging catheter. First, at step 205, insert guidewire into vasculature to a target site. Next, at step 210, advance U/S transducer, sheath and stowed filter through vasculature along guidewire. Next, at step 215, evaluate real time ultrasound image data from the U/S transducer to assess the target site. Next, at step 220, determine filter position within vasculature using ultrasound image data with one or more markings associated with the filter. Next, at step 225, is filter located in appropriate position relative to the target site? If the answer to step 225 is “NO” then proceed to perform one or both of steps 215 and 220 until the answer to step 225 is “YES”. If or when the answer to step 225 is “YES” then proceed to step 230 and deploy the filter. Next, at step 235, assess or confirm degree of filter deployment, position of filter relative to the target site or location of the filter within the vasculature using the ultrasound image data and/or one or markings associated with the filter. Thereafter, continue to step 240 and determine whether or not the filter placement is satisfactory. If the answer to step 240 is ‘NO’ then proceed to step 250 to recapture, reposition or adjust the filter position. Thereafter, return to step 235 and assess the filter deployment. If the answer to step 240 is “YES”, then proceed to withdraw U/S transducer sheath/catheter leaving filter in place. Once complete the method ends at step 255. Once complete the method ends at step 255.

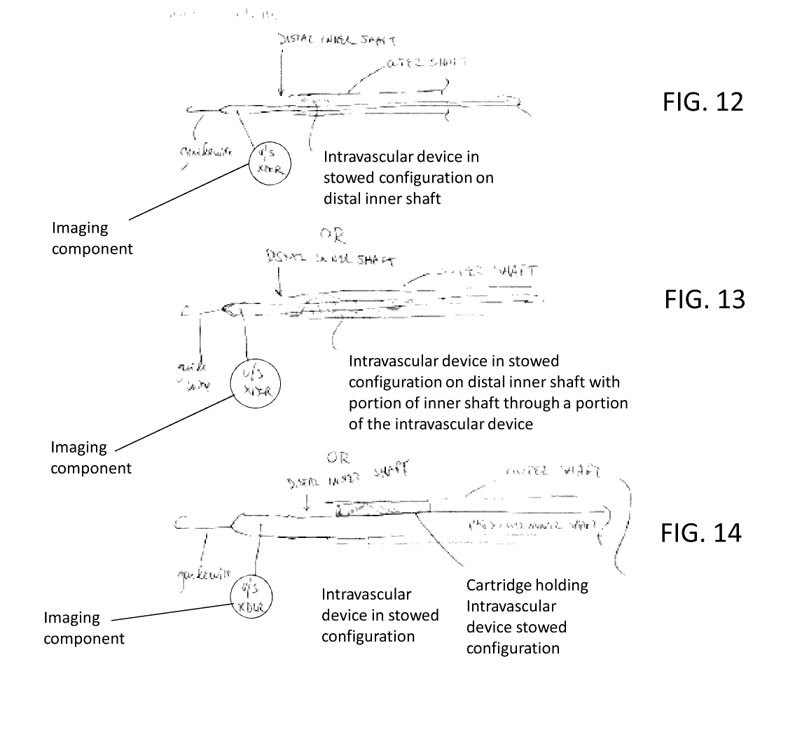

FIG. 12 is a cross section view of an exemplary loading configuration of an intravascular device in a stowed configuration relative to the distal inner shaft of an imaging component catheter embodiment.

FIG. 13 is a cross section view of an exemplary loading configuration of an intravascular device in a stowed configuration relative to the distal inner shaft of an imaging component catheter embodiment whereby the distal inner shaft passes through a portion of the intravascular device here illustrated as a portion of the inner shaft within one or more spiral frames of a filter.

FIG. 14 is a cross section view of an exemplary loading configuration of an intravascular device in a stowed configuration relative to the distal inner shaft of an imaging component catheter embodiment whereby the intravascular device is in a stowed configuration within a separate storage cartridge alongside the distal inner shaft.

In various specific embodiments, FIGS. 12, 13 and 14 may be configure to represent various cross section views of a filter loaded onto an ultrasound catheter as in FIGS. 2A and 2B. Each alternative view represents an inferior vena cava filter in position adjacent to an embodiment of the intravascular ultrasound tracking and filter delivery catheter. In one aspect, FIG. 12 is a cross section view of an exemplary method of loading a filter on the distal inner shaft of an integrated IVUS delivery catheter. In one aspect, FIG. 13 is a cross section view of an exemplary method of loading after onto the distal inner shaft of an integrated IVUS delivery catheter whereby a portion of the inner shaft is within one or more spiral frames of the filter. FIG. 14 is a cross section view of an exemplary method of loading a filter within a separate storage cartridge alongside the distal inner shaft of an integrated IVUS delivery catheter.

In still further additional embodiments, each of the embodiments of FIGS. 12, 13 and 14, the filter may be any of the filters described in International PCT Publication No. WO 2013/106746 A2, titled “ENDOLUMINAL FILTER WITH FIXATION;” International PCT Publication No. WO 2014/152217 A1, titled “ENDOLUMINAL FILTER HAVING ENHANCED ECHOGENIC PROPERTIES;” and International PCT Publication No. WO 2014/152365 A2 titled “FILTERS WITH ECHOGENIC CHARACTERISTICS,” each of which are herein incorporated by reference in its entirety. It is to be appreciated the filter being deployed using the systems and methods described herein may be modified according to the devices, systems, techniques and methods described in “ENDOLUMINAL FILTER HAVING ENHANCED ECHOGENIC PROPERTIES” U.S. Provisional Patent Application Ser. No. 62/054,844 filed Sep. 24, 2014, incorporated herein by reference for all purposes. Additionally or alternatively, the systems, techniques and devices described herein may be secured or controlled using the methods, devices or techniques described in U.S. Provisional Patent Application No. 61/919,573, titled “SECUREMENT DEVICE FOR CONTROLLED ENDOLUMINAL FILTER DEPLOYMENT,” filed Dec. 20, 2013 and U.S. patent application Ser. No. 14/578,087, titled “DEVICES AND METHODS FOR CONTROLLED ENDOLUMINAL FILTER DEPLOYMENT,” filed Dec. 19, 2014 incorporated herein by reference.

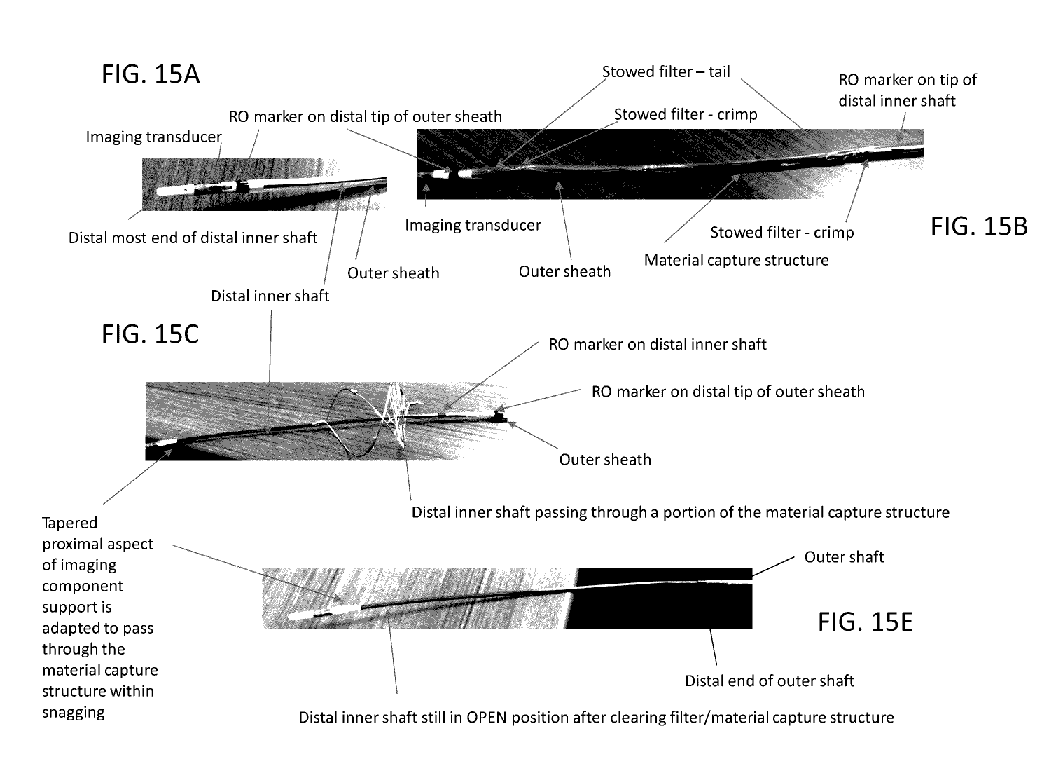

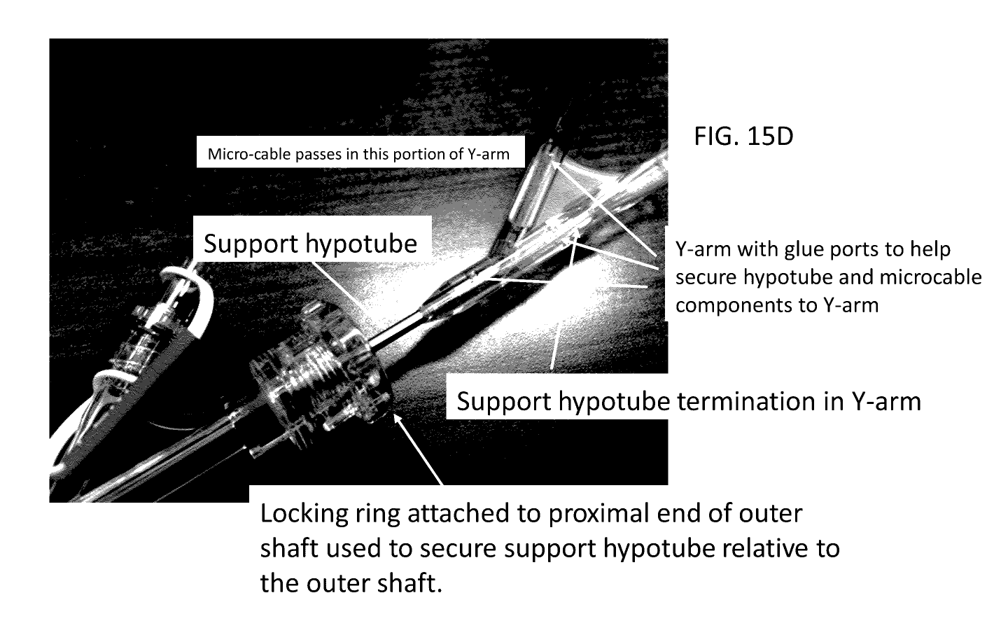

FIG. 15A is a perspective view of the distal end of an integrated ultrasound transducer filter delivery catheter where the handle is in an open configuration and the filter is in a stowed configuration. FIG. 15B is a perspective view of a more proximal portion of the distal end shown in FIG. 15A of the integrated ultrasound transducer filter delivery catheter where the filter and marker relationship is illustrated with the handle remaining in an open configuration and the filter is in a stowed configuration. FIG. 15C is a perspective view of the distal end shown in FIGS. 15A and 15B of the integrated ultrasound transducer filter delivery catheter where the handle has been closed (FIG. 15D) to permit movement between the distal inner tube and the outer tube and transition the filter from stowed to deployed configuration. Also illustrated in the portion of the distal inner tube that remains through a portion of the filter. The distal inner tube is shown extending through a material capture structure and support frame.

FIG. 15D is a perspective view of the handle at the proximal end of the catheter in the condition of FIG. 15C (filter transitioned from stowed to deployed configuration). FIG. 15E is a perspective view of the integrated imaging delivery catheter of FIG. 15C after withdrawal of the imaging capsule through the filter.

In some embodiments of the integrated image guided delivery catheter there is a bleed back valve provided along the inner shaft. In one aspect, there is a torus shaped one way valve (e.g. porous membrane or mechanical seal) can be incorporated in between the outer shaft and inner shaft lumen of the device, just distal of the printed linear markings. The valve should open towards the distal end of the device and seal shut towards the proximal end. This valve would allow the user to flush the lumen between the two shafts from the handle side port, but restrict liquids from flowing pass it and covering the linear markings on the proximal inner shaft during use.

In some embodiments, the handle or Y-arm hub is configured for an additional side port/lumen for contrast injection. For this design, another lumen maybe incorporated into the body of the catheter, and a third luer port can be added onto the Y-Arm design described above. The additional lumen maybe lined with material optimized for contrast injection. During use, contrast will be injected through the third Y-Arm luer port and exit on the distal end of the device near the transducer.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An intravascular imaging capsule, comprising:
an imaging component mounted on a support frame with a proximal end and a distal end having a base adapted and configured to at least partially encircle a support tube, a proximal facing shoulder mount and a distal facing shoulder mount;
a distal tip having a distal end and a proximal end and a lumen extending from the proximal end to the distal end wherein a proximal portion of the lumen is sized to fit over a portion of the distal facing shoulder mount and a distal portion of the lumen is sized to have an inner diameter of about the same size as an inner diameter of the support tube; and
proximal cap having a distal end and a proximal end and a lumen extending from the proximal end to the distal end sized to be greater than the largest cross section dimension of the support tube wherein a distal portion of the lumen is sized to fit over a portion of the proximal facing shoulder mount.

2. The capsule of claim 1 wherein the imaging component is one of a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT).

3. The capsule of claim 1 wherein an exterior portion of the proximal cap is scalloped to accommodate a portion of the imaging component.

4. The capsule of claim 3 wherein the scalloped portion of the proximal cap further comprising a pass through formed in a scalloped wall of the proximal cap extending from an opening in a proximal cap exterior wall to an opening in communication with the proximal cap lumen, wherein the opening in the proximal cap exterior wall is located distal to the opening in communication with the proximal cap lumen.

5. The capsule of claim 3 wherein the scalloped portion of the proximal cap further comprising a pass through formed in a scalloped wall of the proximal cap extending from an opening in a proximal cap exterior wall to an opening in communication with the proximal cap lumen, wherein the pass through forms an angle of about 45 degrees relative to the proximal cap lumen.

6. The capsule of claim 3 wherein the portion of the imaging component is an electronic connector for communication with an image processing system configured for use with the imaging component.

7. The capsule of claim 6 wherein the electronic connect comprises a flexible leg extending from the support frame.

8. The capsule of claim 1 the proximal cap further comprising a pass through formed in a wall of the proximal cap extending from an opening in a proximal cap exterior wall to an opening in communication with the proximal cap lumen, wherein the opening in the proximal cap exterior wall is located distal to the opening in communication with the proximal cap lumen.

9. The capsule of claim 1 the proximal cap further comprising a pass through formed in a wall of the proximal cap extending from an opening in a proximal cap exterior wall to an opening in communication with the proximal cap lumen, wherein the pass through forms an angle of about 45 degrees relative to the proximal cap lumen.

10. The capsule of claim 4 wherein the pass through or opening is sized to pass a microcable for connection of the imaging component to an image processing system configured for use with the imaging component.

11. The capsule of claim 1 further comprising a proximal portion of the proximal cap lumen sized to have an inner diameter to accommodate the support tube, a sheath over the support tube dimensioned to provide a gap between the sheath and the support tube to allow passage of a cable; and
the sheath over the support tube is attached to an interior wall of a portion of the proximal lumen.

12. The capsule of claim 11 wherein the base completely encircles the support tube.

13. The capsule of claim 12 wherein the base has a cross section shape similar to a cross section shape of the support tube.

14. The capsule of claim 13 wherein the cross section shape of the support tube is circular.

15. The capsule of claim 1 comprising glue holes positioned to allow centering in the distal tip so that they can fill a gap between the support tube and an interior wall of the distal tip.

16. The capsule of claim 15 wherein the glue holes are across a central longitudinal axis of the distal tip lumen.

17. The capsule of claim 1 wherein the distal tip has a tapered shape from the proximal end to the distal end.

18. The capsule of claim 1 wherein the distal most end of the distal tip is a rounded atraumatic shape.

19. An image guided intravascular device delivery catheter, comprising:
a support tube with proximal end and a distal end and a lumen extending therethrough;
a handle or y-valve having an aperture adapted to receive the proximal end of the support tube so that the lumen of the tube is coextensive with a lumen of the handle in communication with the handle aperture adapted to receive the support tube proximal end whereby movement of the handle produces movement of the support tube;
a distal inner shaft having a proximal end and a distal end with a single lumen extending from the proximal end to the distal end sized to receive the support tube;
a proximal inner shaft having a proximal end and a distal end with a first lumen and a second lumen each extending from the proximal end to the distal end wherein the first lumen is larger than the second lumen and the first lumen is sized to receive the support tube;
an inner shaft marker attached to the proximal most end of the distal inner shaft or distal most part of the proximal inner shaft wherein the inner shaft maker is positioned to indicate the transition from the distal inner shaft to the proximal inner shaft;
an outer shaft having a proximal end and a distal end and a lumen there through sized to receive the proximal inner shaft;
an outer shaft marker positioned at the distal portion of the outer shaft;
a locking device on the proximal portion of the outer shaft distal to the handle, the locking device having a locked configuration that impedes relative movement between the outer shaft and the handle and an unlocked configuration that permits relative movement between the outer shaft and the handle;
an intravascular image tracking capsule having an imaging component supported between a distal tip and a proximal cap wherein the distal tip, the imaging component and the proximal cap are arranged about a common lumen that is adapted to receive the support tube distal end; and
an intravascular device positioned between an exterior portion of the distal inner shaft and an interior portion of the outer shaft wherein while the outer shaft marker is positioned distal to the inner shaft marker movement between the intravascular device and the distal inner shaft is restricted.

20. The device of claim 19 further comprising a plurality of spaced apart distance markers along the outer shaft.

21. The device of claim 20 wherein the plurality of spaced apart markers are printed on a surface of the outer shaft.

22. The device of claim 20 wherein the plurality of spaced apart markers indicate a distance relative to the intravascular device.

23. The device of claim 19 wherein when the outer shaft marker is positioned proximal to the distal inner shaft marker relative movement between the intravascular device and the distal inner shaft is permitted.

24. The device of claim 19 wherein when the outer shaft marker is positioned proximal to the distal inner shaft marker the intravascular device is in a deployed configuration.

25. The device of claim 19 wherein when the outer shaft marker is positioned proximal to the distal inner shaft marker the handle is adjacent to the locking device.

26. The device of claim 19 wherein the axial distance moved by the outer shaft marker when moved from a position distal to the inner shaft marker to a position proximal to the inner shaft marker is greater than an axial length of the intravascular device when positioned between an exterior portion of the distal inner shaft and an interior portion of the outer shaft.

27. The device of claim 19 wherein a proximal portion of the intravascular imaging capsule proximal cap is adapted and configured to mitigate engagement with the intravascular device.

28. The device of claim 19 wherein the intravascular imaging capsule proximal cap has a tapered portion adjacent to where the support tube exits the intravascular imaging capsule.

29. The device of claim 19 wherein the intravascular imaging capsule is sized to pass through a portion of the intravascular device.

30. The device of claim 29 wherein the intravascular device is a filter and the portion of the intravascular device is a filtering component or a material capture structure.

31. The device of claim 19 wherein the intravascular device includes one or more elements configured for detection by the imaging component.

32. The device of claim 31 wherein the one or more elements are positioned on, about, along, around, or within one or more portions of the intravascular device such that when the one or more elements are detected by the imaging component an output of the imaging component includes an indication of a position, an orientation, a state of deployment, a state of retrieval, a state of operation, or a condition of the intravascular device.

33. The device of claim 19 wherein the imaging component is one of a single crystal piezoelectric transducer, a ceramic piezoelectric transducer, an optical coherence tomography element, an intravascular ultrasound transducer, a rotational intravascular ultrasound transducer, a piezo-electric micro-machined ultrasonic transducer (PMUT), and a capacitive micro-machined ultrasonic transducer (CMUT).

34. The device of claim 33 further comprising a communication cable connected at one end to the imaging element and at the other end to a connector adapted and configured for use with an image processing system configured for use with the imaging element, the communication cable passing through a lumen of the handle, the second lumen of the proximal inner shaft, along an outer surface of the distal inner shaft, and through an opening formed in the sidewall of the proximal cap.

35. The device of claim 34 wherein the opening formed in the sidewall of the proximal cap is angled towards the proximal portion of the distal inner shaft.

36. The device of claim 35 wherein the opening formed in the sidewall of the proximal cap forms an angle of about 45 degrees relative to an exterior wall of the proximal cap.

37. The device of claim 35 wherein the opening formed in the sidewall of the proximal cap is positioned in a portion of the proximal cap shaped to correspond to a portion of a flexible component coupled to the imaging element.

38. The device of claim 37 wherein the portion of the proximal cap shaped to correspond has a smaller outer diameter compared to a directly adjacent portion of the proximal cap.

39. The device of claim 37 wherein the portion of the proximal cap shaped to correspond has a flat surface that is sized to approximate a portion of the flexible component.
